(19) United States
(12) Patent Application Publication
(10) Pub. No.: US 2013/0142693 A1
(43) Pub. Date: Jun. 6, 2013

(54) PHOTOCATALYST FOR OXIDATION REDUCTION CHEMISTRY

(71) Applicant: Earth Tone Energies, LLC, Moorhead, MN (US)

(72) Inventor: Timothy J. Flick, Moorhead, US (US)

(73) Assignee: EARTH TONE ENERGIES, LLC, Moorhead, MN (US)

(21) Appl. No.: 13/685,269

(22) Filed: Nov. 26, 2012

Related U.S. Application Data
(63) Continuation-in-part of application No. 12/814,238, filed on Jun. 11, 2010, now abandoned.

(51) Int. Cl. A61L 2/18

(52) U.S. Cl. CPC A61L 2/18
USPC 422/29; 422/186.3; 422/186; 423/610; 502/350

(57) ABSTRACT

Embodiments of the present disclosure combine a suitable photocatalyst with a non-conducting matrix such as plastic, glass or rubber for the purpose of the production of activated electrons, needed in the creation of hydrogen peroxide, in the presence of light of a suitable frequency or frequencies and water. A suitable photocatalyst such as anatase titanium dioxide is combined with a plastic such as polypropylene as one would a pigment. The impregnated plastic can be immersed in water whereupon activated electrons and holes (electron absences in the valence band of the plastic substrate acting as a semiconductor) are produced on the surface of the photocatalyst upon irradiation. Activated electrons are an excellent oxidizer, disinfectant, purifier and go on to kill bacteria, algae, etc. in the water, as well as to reduce water hardness including mineral deposits. Unused hydrogen peroxide breaks down into hydrogen ion and free oxygen in a short time.

### This is a continuation-in-part application of application Ser. No. 12/814,238, filed Jun. 11, 2010.

### FIELD OF THE DISCLOSURE

Embodiments of the present disclosure generally relate to substantially cleaning impure water. Particularly, embodiments of the present disclosure relate to disinfecting apparatus. More particularly, embodiments of the present disclosure relate to disinfectant systems for the efficient disinfection of contaminated water.

### BACKGROUND

Contaminants within fluid sources (e.g., both gas and liquid state) and surfaces are prevalent and can cause great amounts of harm to those animals or plants in contact with the contaminants. Various types of disinfectants and filtering devices have been used in the past to try to rid the contaminants from the fluid sources and surfaces.

However, these disinfectants and filtering devices generally do not work properly, by not ridding the fluid source/surfaces of the contaminants and adding further pollutants to the fluid source/surfaces. Decontamination using disinfectants and filtering devices can be very time consuming, requiring constant attention, or simply too costly for small production facilities or reservoir structures, such as livestock water tanks, pools or toilets.

There have been methods suggested for the use of titanium dioxide in the anatase form for use in ceramics for producing self disinfecting surfaces. The main drawback is the high working temperatures for ceramic substrates. These would require acidic water to work properly, as well. There have also been reported plastics with antibodies engineered into their matrix to produce antibacterial surfaces, but the process is expensive and selective for certain microorganisms.

Because of the inherent problems with the related art, there is a need for a new and improved disinfectant system for the efficient disinfection of contaminated surfaces and fluids. It would be desirable to find a water purification system where no fossil fuel is needed for sustained operations; disinfection and softening of questionable drinking water is provided; the system is gravity fed requiring no pumps; there are no residual carcinogenic, otherwise toxic or ecologically harmful by products; precise monitoring can be possible, giving the ability to adjust for the amount of hardness in the feed water; and the water has a pleasant taste.

### SUMMARY

Embodiments of the present disclosure combine a suitable photocatalyst with a non-conducting matrix such as plastic, glass or rubber for the purpose of the production of activated electrons, needed in the creation of hydrogen peroxide, in the presence of light of a suitable frequency or frequencies and water. A suitable photocatalyst such as anatase titanium dioxide is combined at low temperature with a plastic such as polypropylene, as one would a pigment. The impregnated plastic can be immersed in water, and activated electrons and holes (electron absences in the valence band of the plastic substrate acting as a semiconductor) are produced on the surface of the photocatalyst upon irradiation. Activated electrons (including the produced hydrogen peroxide) are an excellent oxidizer, disinfectant, purifier, and go on to kill bacteria, algae, etc. in the water, as well as to reduce water hardness caused by mineral deposits such as iron. Unused hydrogen peroxide breaks down into hydrogen ion and free oxygen in a short time.

### BRIEF DESCRIPTION OF THE DRAWINGS

The above and other objects, features and other advantages according to several embodiments of the present disclosure will be more clearly understood from the following detailed description taken in conjunction with the accompanying drawings, in which:

FIG. 1 is an upper perspective view of an embodiment of the present disclosure within a reservoir structure comprised of a livestock water tank;

FIG. 2 is an upper perspective view of an embodiment of the present disclosure;

FIG. 3 is an upper perspective view of an embodiment of the present disclosure with the float exploded outwards;

FIG. 4 is a top view of an embodiment of the present disclosure;

FIG. 5 is a side sectional view taken along lines 5-5 of FIG. 4;

FIG. 6 is a side cross-sectional view of an embodiment of the present disclosure within a fluid source;

FIG. 7 is an illustrative cross-sectional view of the carrier showing the photocatalyst evenly distributed throughout the substrate material in an embodiment of the present disclosure;

FIG. 8 is an illustrative cross-sectional view of the carrier showing the treatment surface continually exposed to an outside of the carrier as the carrier degrades during use in an embodiment of the present disclosure;

FIG. 9 is an upper perspective view of the structure comprised of a livestock water tank functions in an embodiment of the present disclosure;

FIG. 10 is a side view of the carrier within a water bottle in an embodiment of the present disclosure;

FIG. 11 is a top sectional view of an embodiment of the present disclosure;

FIG. 12 is a top view of the carrier within urinal in an embodiment of the present disclosure;

FIG. 13 is an upper perspective view of an embodiment of the present disclosure;

FIG. 14 is an upper perspective view of an embodiment of the present disclosure;

FIG. 15 is an upper perspective view of embodiments of the present disclosure positioned over an oil spill on the ground surface;

FIG. 16 is an upper perspective view of embodiments of the present disclosure;

FIG. 17 is a front profile view of an embodiment of the present disclosure for a purification system for unclean water;

FIG. 18 is a front profile view of an embodiment for an injector system in the present disclosure; and

FIG. 19 is a front profile view of a citric acid dispenser in an embodiment of the present disclosure;

While the improved photocatalyst for oxidation reduction chemistry is amenable to various modifications and alternative forms, specifics thereof have been shown by way of example in the drawings and will be described in detail. It should be understood, however, that the intention is not to limit an improved photocatalyst for oxidation reduction chemistry to the particular embodiments described. On the contrary, the improved photocatalyst for oxidation reduction chemistry is to cover all modifications, equivalents, and alternatives.

### DETAILED DESCRIPTION

Hereinafter, embodiments of the present disclosure will be described in detail with reference to the accompanying drawings.

The following discussion is presented to enable a person skilled in the art to make and use the present disclosure. Various modifications to the illustrated embodiments will be readily apparent to those skilled in the art, and the generic principles herein may be applied to other embodiments and applications without departing from the present teachings. Thus, the present teachings are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the present disclosure. Skilled artisans will recognize the examples provided herein have many useful alternatives and fall within the scope of the present teachings.

Embodiments of the present disclosure disclose a system for the efficient disinfection of contaminated surfaces and fluids. Embodiments of the present disclosure generally relate to a disinfecting apparatus, which includes a light source for producing ultraviolet light, a fluid source containing organic contaminants and a carrier comprising a substrate material and a photocatalyst.

The photocatalyst is evenly distributed throughout the substrate material so a treatment surface of the carrier is continually exposed to the fluid source and the ultraviolet light as the substrate material degrades. For the purposes of this disclosure, “evenly distributed throughout”, in regards to the photocatalyst being evenly distributed throughout the substrate material, is defined to mean that the photocatalyst is found in approximately equal distribution in the substrate material including on the surface and internally. The substrate material comprises an electrically non conductive material. The treatment surface is positioned at least partially within the fluid source and wherein the ultraviolet light is focused upon the treatment surface for oxidizing the organic contaminants within the fluid source. The carrier may be used for various purposes such as for disinfecting drinking water, ground surfaces, and table surfaces. The carrier may also be supported in various frames or support structures.

For the purposes of this disclosure, “organic contaminants”, in regards to the fluid source containing organic contaminants, is defined as a material having a carbon-basis including chemicals such as solvents, pesticides, and polychlorinated biphenyls (PCB's) and organic plant matter such as bacteria, algae, oils, etc.

The inventor was performing experiments on an inexpensive production method for the production of activated electrons necessary for the creation of hydrogen peroxide when it became apparent hydrogen peroxide would be a good disinfection method for producing potable water. Embodiments of the present disclosure involve the use of very inexpensive ingredients to produce a high benefit to cost ratio. It involves relatively low temperature production methods allowing titanium dioxide to remain in the anatase form throughout the production process. It also allows for an extended working lifetime since the photocatalyst can be distributed throughout the substrate material. As the treatment surface is sloughed off, new catalyst is exposed.

Embodiments of the disclosure comprise a float attached to the center of either a square or circular flat plastic backing and impregnated grid assembly, or a flat plastic impregnated matrix without backing. These units are central to support accessories such as acidifiers, tanks, filters, plumbing and sensors with controller(s).

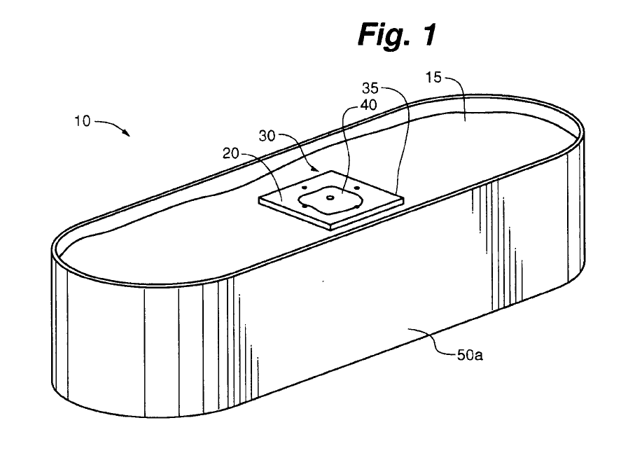

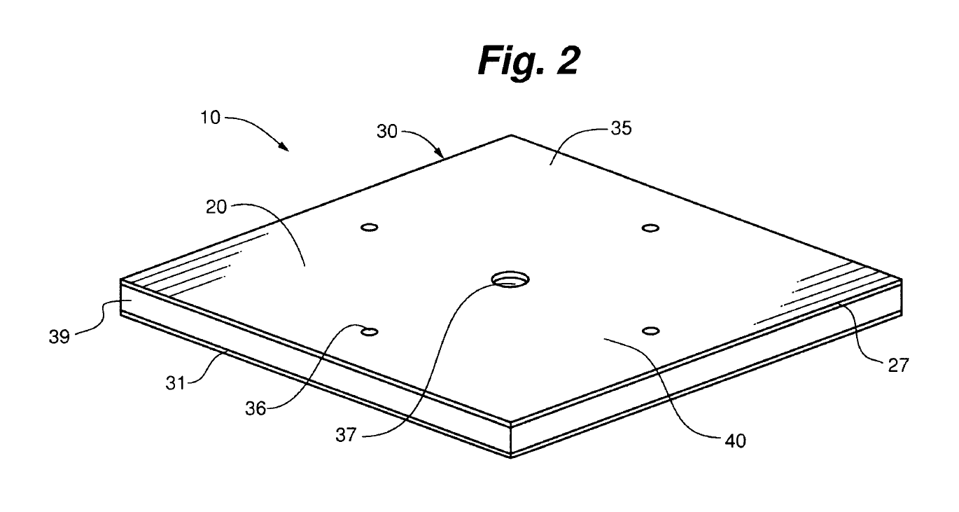

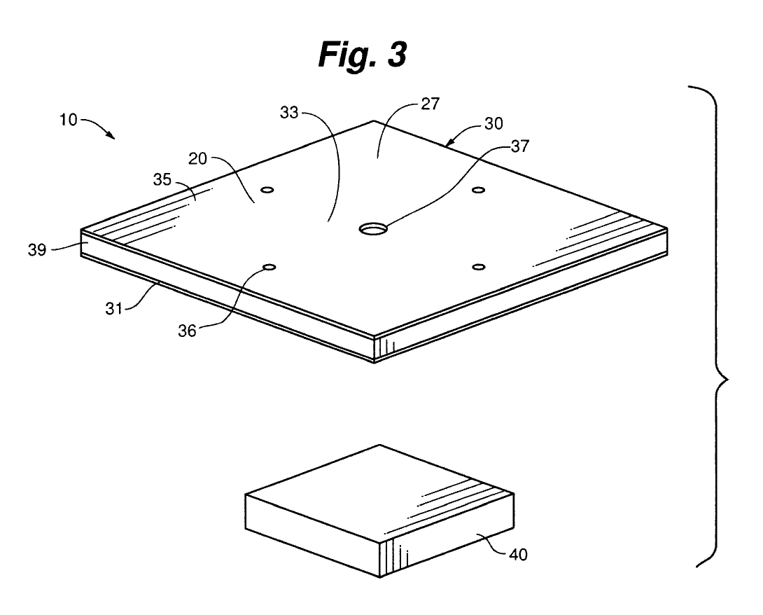

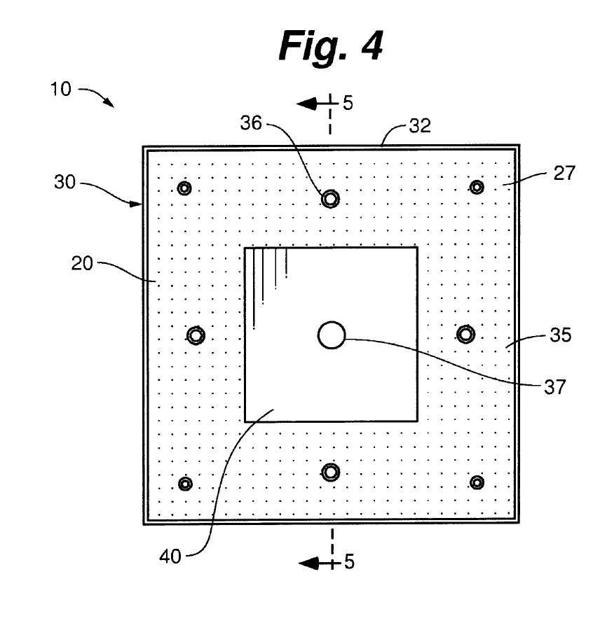

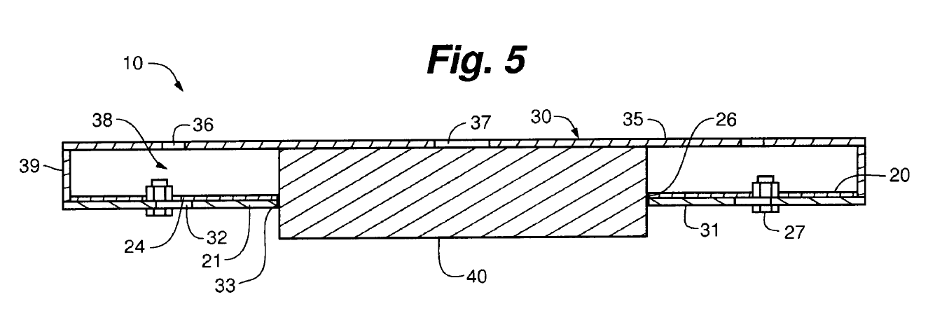

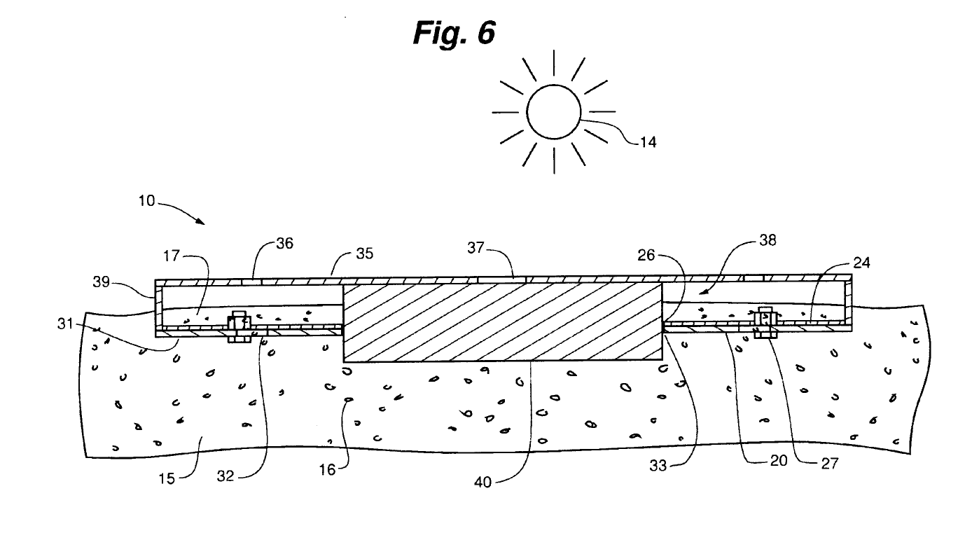

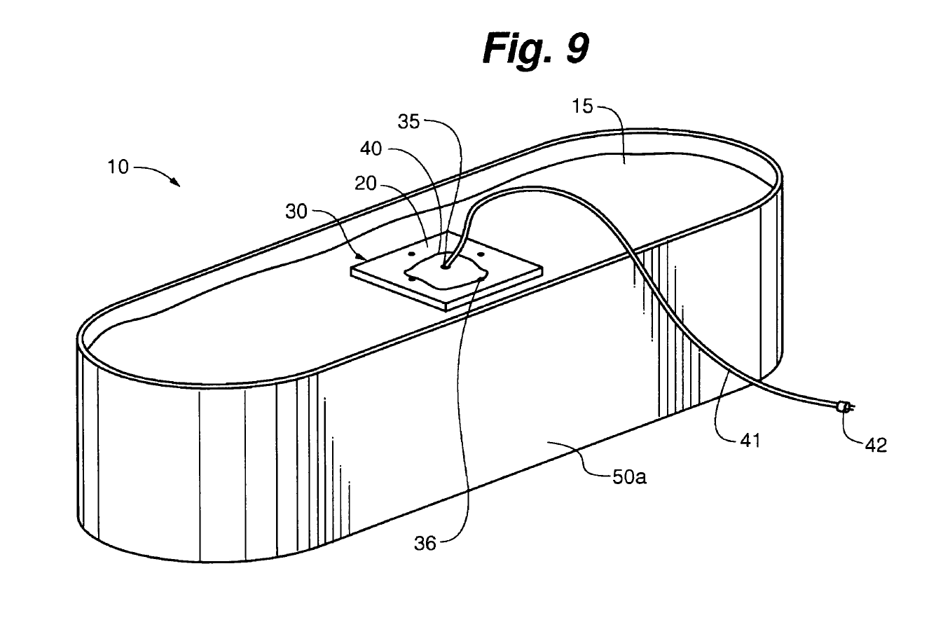

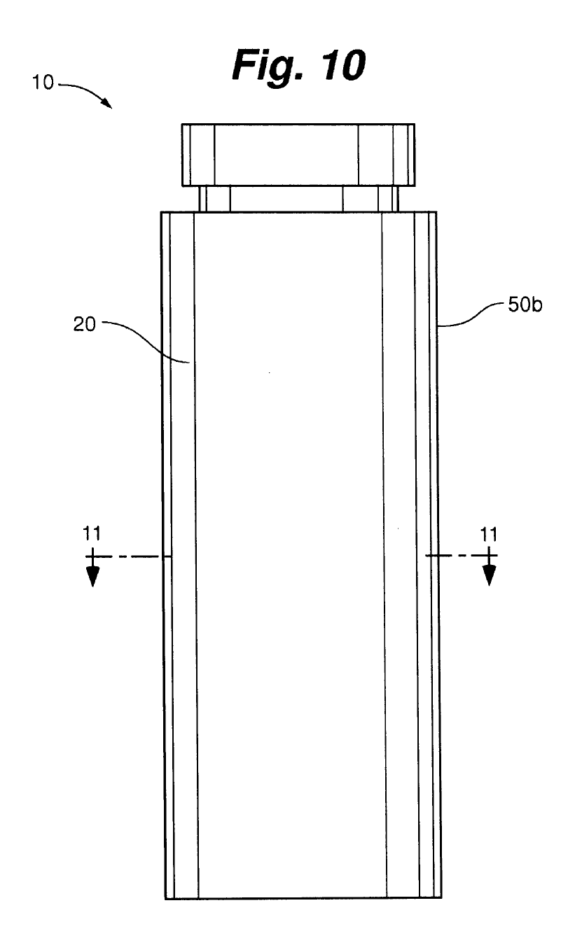

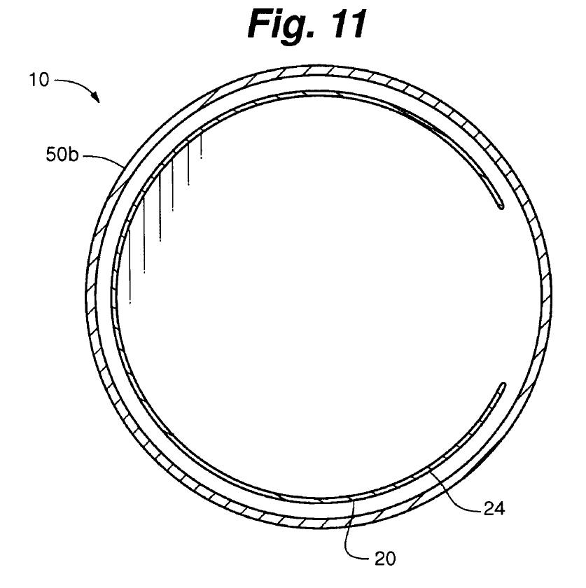

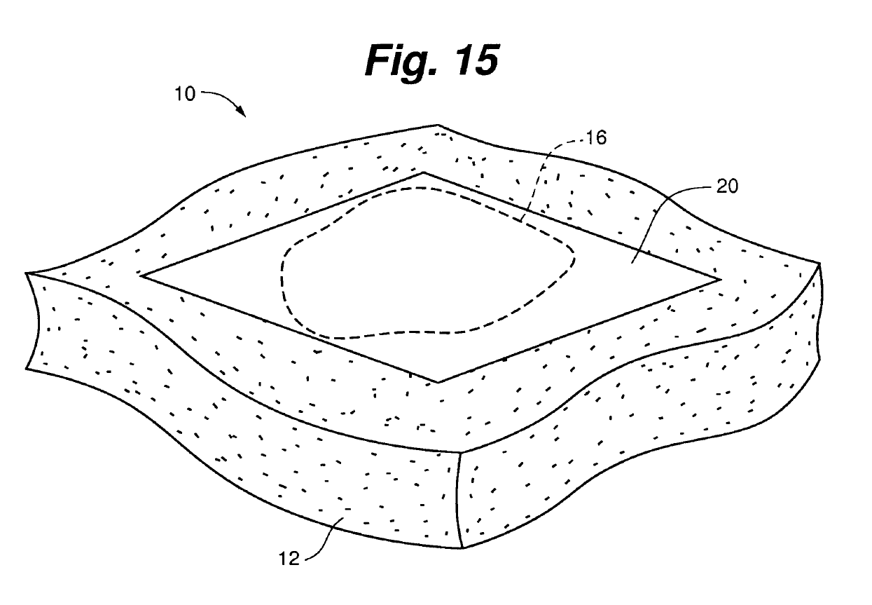

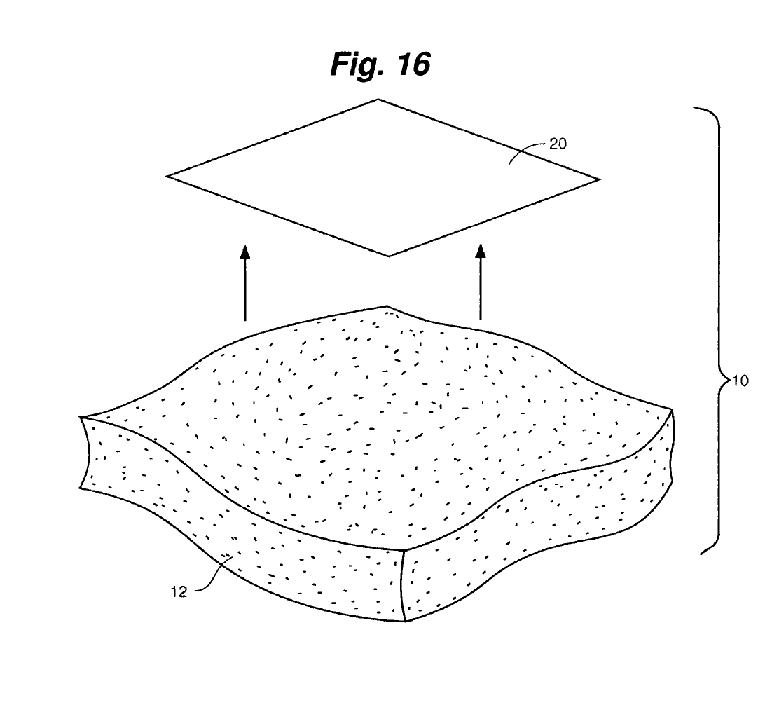

Turning now to the drawings, in which similar reference characters denote similar elements throughout the several views, FIGS. 1 through 16 illustrate a disinfectant system 10, which comprises a light source 14 for producing ultraviolet light, a fluid source 15 having organic contaminants 16 within and a carrier 20 comprising a substrate material 21 and a photocatalyst 22. The photocatalyst 22 is evenly distributed throughout the substrate material 21 so a treatment surface 24 of the carrier 20 is continually exposed to the fluid source 15 and the ultraviolet light as the substrate material 21 degrades. The substrate material 21 is comprised of an electrically non conductive material. The treatment surface 24 is positioned at least partially within the fluid source 15 and wherein the ultraviolet light is focused upon the treatment surface 24 for oxidizing the organic contaminants 16 within the fluid source 15. The carrier 20 may be used for various purposes, such as for disinfecting drinking water, ground surfaces 12, table surfaces and other contaminated objects. The carrier 20 may also be supported in various frames 30 or support structures.

The fluid source 15 may refer to various types of fluids, such as a fluid source in a liquid state (e.g. water, etc.), a fluid source in a gaseous state (e.g. air), or a combination. For example, the liquid state may come into use when the carrier 20 is used within a reservoir structure 50a, 50b, 50c, such as a livestock tank as illustrated in FIGS. 1 through 12.

The light source 14 may also refer to various types of lights, such as a light source comprising the sun, a light source comprising ultraviolet light bulbs, or other ambient light sources. It is appreciated a partially obstructed light source 14 may also be used with the carrier 20. The ultraviolet light produces highly reactive forms of oxygen (oxygen free radicals and hydrogen peroxides) in the oxygenated fluid source 15 contributing to the destruction process of the microorganisms or organic contaminants 16 into oxidized particles 17.

The carrier 20 is used to oxidize the organic contaminants 16 within the fluid source 15 through a photocatalytic reaction between the carrier 20, ultraviolet light and the fluid source 15, wherein the fluid source 15 includes hydrogen elements and oxygen elements. The carrier 20 induces a chemical reaction to form activated electrons which are necessary in the creation of low amounts of hydrogen peroxide to break down the contaminants 16 into oxidized particles 17 and thus effectively disinfect the fluid source 15 with the hydrogen peroxide. The carrier 20 may take the form of various shapes and configurations to fit within various size frames 30, other reservoir structures 50a, 50b, 50c, or be placed upon the ground surface 12 or various other objects as desired, whatever location has the need to disinfect or decontaminate. The carrier 20 is also substantially inert in that the carrier 20 does not move during the chemical reaction, except the slight degrading of the substrate material 21. The carrier 20 itself can comprise a buoyant structure to float so the carrier 20 may be placed within various fluid sources 15 and efficiently oxidize contaminants 16 near the surface of the fluid source 15.

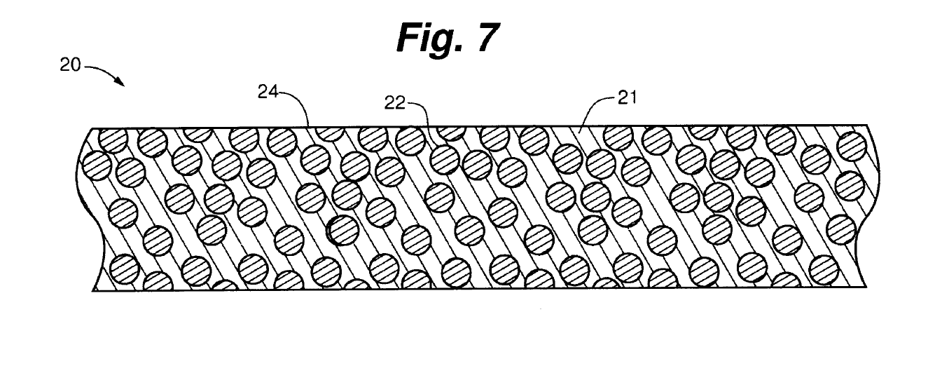

In one embodiment, the carrier 20 comprises a substrate material 21 and a photocatalyst 22 material incorporated within. The treatment surface 24 includes the portion of the carrier which has the photocatalyst 22 mixed with the substrate material 21. The treatment surface 24 and photocatalyst 22 can be distributed evenly throughout the entire substrate material 21 and thus entire carrier 20 as illustrated in FIG. 7. However, in alternate embodiments, the treatment surface 24 may be instead along the perimeter walls of openings extending through the carrier 20 (in the mesh shape), upon a top surface, a bottom surface, or portions thereof. The treatment surface 24 may simply be a small portion of the substrate material 21 or carrier 20, of which contacts the fluid source 15 and receives the ultraviolet light from the light source 14. The substrate material 21 may also comprise a permeable and absorbent structure so the contaminants 16 can travel within the carrier 20 to be oxidized within. It is appreciated various combinations of the above described, as well as other combinations, may also be used to combine the photocatalyst 22 with the substrate material 21.

The substrate material 21 can comprise an electrically non conductive material, such as a plastic, which includes rubber, polystyrene, polymers, nylon, polyethylene, acrylic or other various types of plastic or non conductive materials and combinations of the various materials (e.g. substrate material 21 comprising rubber and polyethylene). The substrate material 21 may also be absorbable to digest the contaminants 16 for the chemical reaction to take place. The use of a non conductive material, such as plastic, is important to provide an economic, variable product which is easy to manufacture in various sizes, shapes and forms. The use of a substrate material 21 comprising plastic also provides a low melting temperature which helps to induce the chemical reaction and thus provide for a more efficient self disinfecting treatment surface 24.

The substrate material 21 is pigmented with the photocatalyst 22 composed of titanium dioxide and has properties to induce a chemical reaction when exposed to ultraviolet light rays from the light source 14. The photocatalyst 22 can be titanium dioxide in the anatase crystalline form rather than its rutile form. After the pigmentation melt process the substrate material 21, including the photocatalyst 22, can be extruded in various forms whose surfaces 24 are photocatalytic in the oxidation of oxygenated water (e.g. fluid source 15) to hydrogen peroxide.

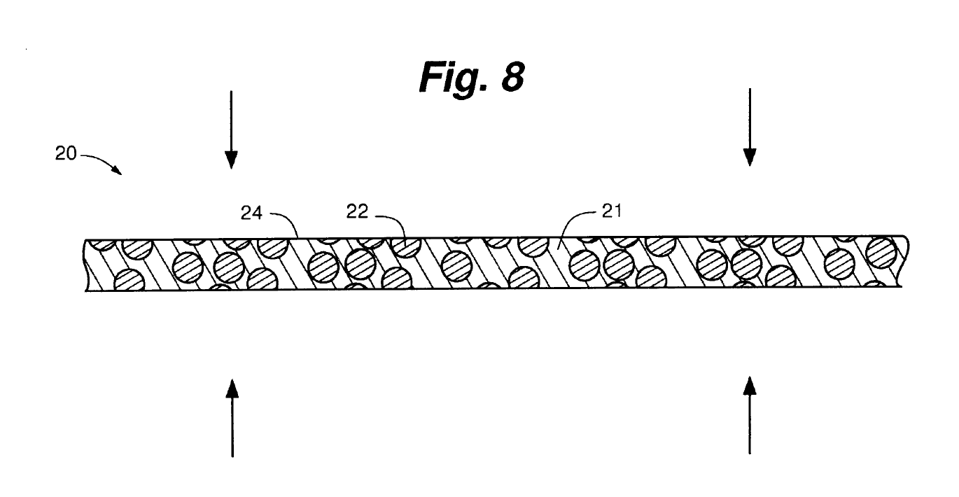

The photocatalyst 22 can be an absorbing substance to be able to absorb the ultraviolet light. When receiving the ultraviolet light, the photocatalyst 22 is able to oxidize the organic contaminants 16 to essentially self-disinfect the fluid source 15 or other type of surface or object. The treatment surface 24 extends throughout the substrate material 21 and thus is continually exposed as the substrate material 21 degrades away from the chemical reaction of the oxygen from the fluid source 15 and the ultraviolet light from the light source 14 to form activated electrons that are necessary to form hydrogen peroxide to break down the contaminants 16 into oxidized particles 17 as illustrated in FIG. 8.

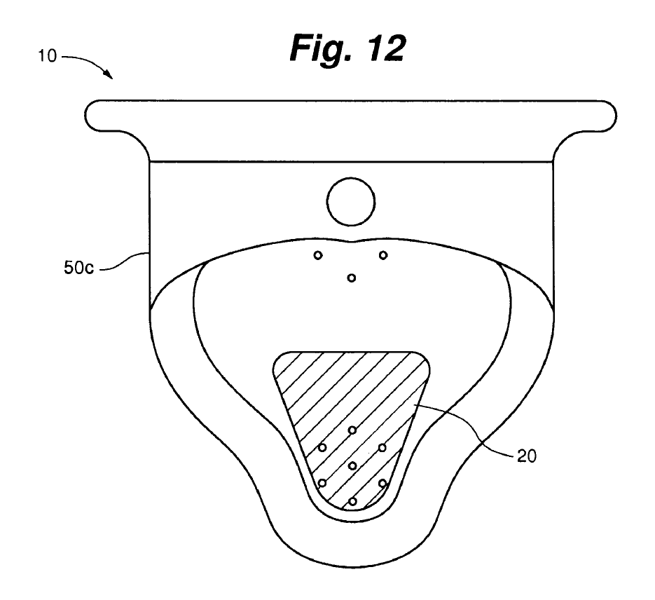

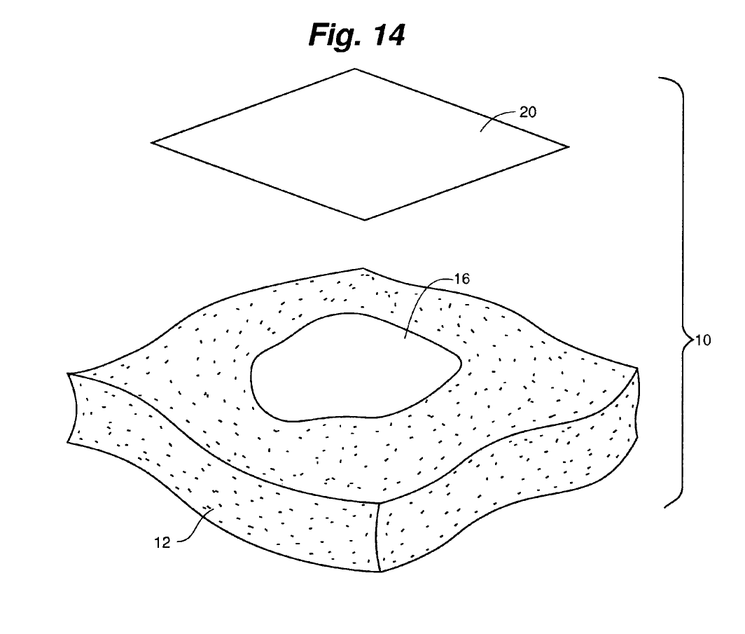

In one embodiment, the carrier 20 is formed into a mesh structure. The mesh structure allows the fluid source 15 to pass through while disinfecting the fluid source 15 by oxidizing the contaminants 16 therein. The mesh carrier 20 may be placed in various locations. One embodiment shows the mesh carrier 20 within the frame 30 for being positioned within a livestock tank as illustrated in FIGS. 1 through 7; another embodiment shows the carrier 20 positioned in a plastic drinking container to disinfect the water therein as illustrated in FIGS. 1, 10 and 11; another embodiment shows the mesh carrier 20 positioned within a urinal over the drainage area to disinfect the urinal as illustrated in FIG. 12, and another embodiment shows the mesh carrier 20 positioned upon a ground surface 12 to oxidize and digest an oil spill area as illustrated in FIGS. 14 through 16. Various other organic contaminant sources may be disinfected with the mesh carrier 20, the mesh carrier 20 may be adapted to various shapes and sizes.

When positioned around the float 40 of the frame 30, in one embodiment of the present disclosure, which will subsequently be described, the carrier 20 may include one or more openings 26 extending therethrough. The carrier 20 may also be secured to the frame 30 or other structure through the use of fasteners 27, such as but not limited to bolts.

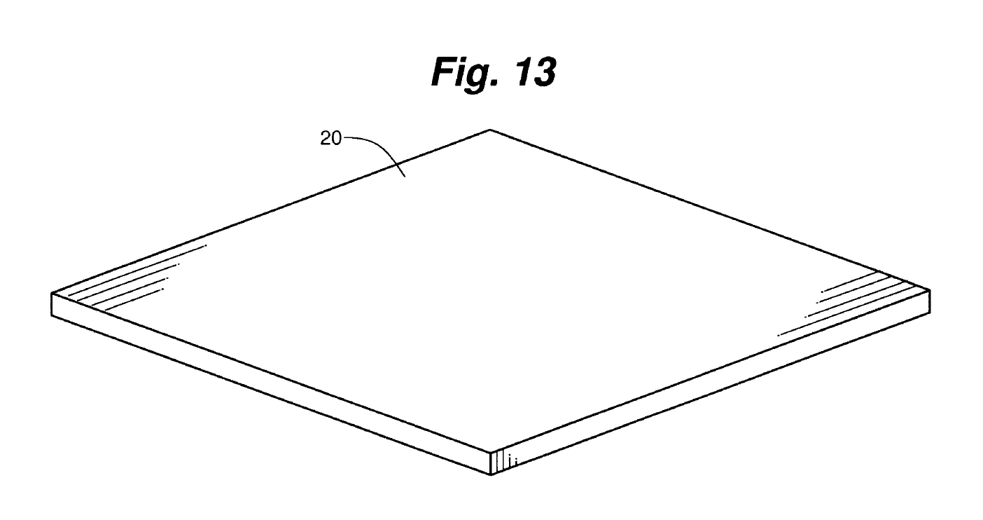

In another embodiment of the carrier 20, the carrier 20 is formed into a cutting board configuration as illustrated in FIG. 13. Since the carrier 20, and substrate material 21 can be made of plastic, the carrier 20 is often molded into its final solid shape. In the case of the cutting board configuration, the carrier 20 is molded into a rectangular or other shaped cutting board. The photocatalyst 22 coating upon the substrate material 21 of the carrier 20 is thus able to disinfect the cutting board surface (i.e. treatment surface 24) to keep the cutting board surface sterile or near sterile and provide a healthier atmosphere in which to serve and prepare food.

In an alternative embodiment to the cutting board configuration, the photocatalyst 22 may be blended with the substrate material 21 prior to molding, allowing for a cutting board surface that continuously disinfects regardless of continued and prolonged use of the board including treatment surface 24 degradation.

In one embodiment of the present invention, the frame 30 is used to support the carrier 20. The frame 30 comprises a rectangular or square shaped structure; however it is appreciated other shapes may be contemplated. The frame 30 is configured to be positioned within a reservoir structure 50a comprising a livestock tank commonly used to hold water for livestock to drink. The carrier 20 in the frame 30 serves to disinfect the water within the reservoir structure 50a thus providing a clean contaminant free water for the livestock.

In an embodiment, the frame 30 includes a lower wall 31 including a plurality of inlets 32 spaced around an inner perimeter and lower receiving opening 33 generally extending through a central portion of the lower wall 31. Sidewalls 39 vertically extend from the outer perimeter of the lower wall 31 and an upper wall 35 is attached to the upper end of the sidewalls 39, thus vertically offsetting the upper wall 35 with respect to the lower wall 31. The upper wall 35 includes a plurality of outlets 36 to substantially align with the inlets 32 of the lower wall 31 and an upper receiver opening 37 also can be near a center of the upper wall 35 similar to the lower receiver opening 33.

The carrier 20 can be affixed to the upper surface of the lower wall 31 and thus within a cavity 38 defined between the upper wall 35 and the lower wall 31. The cavity 38 can be substantially larger in height than the carrier 20 to allow room for the oxidized particles 17 to escape through the outlets 36 of the upper wall 35. The carrier 20 may be affixed to the lower wall 31 in various manners, such as through the use of the fasteners 27 (e.g. bolts, etc.) or other securing mechanisms.

The treatment surface 24 of the carrier 20 can be positioned directly over the inlets 32 so the contaminants 16 can easily engage the treatment surface 24 and thus be oxidized by the photocatalytic reaction. A plurality of inlets 32 may extend through the lower wall 31 so the fluid source 15 having the contaminants 16 may engage the carrier 20 in a plurality of different locations. Once the contaminants 16 are oxidized by the photocatalytic reaction, the oxidized particles 17 can escape the cavity 38 through the outlets 36 of the upper wall 35.

The frame 30 and at least the upper wall 35 also comprise a transparent configuration to allow the ultraviolet light from the light source 14 to pass through and be focused upon the treatment surface 24 of the carrier 20. The upper wall 35 also serves another purpose, besides providing support for the frame 30, which is to protect the carrier 20 by preventing the livestock or foreign particles from engaging or contacting the carrier 20. The upper wall 35 and thus sidewalls 39 extend over and surround the entire carrier 20 besides the portion of the carrier 20 is accessible through the inlets 32 and outlets 36. However, the inlets 32 and outlets 36 are substantially small, wherein only contaminants 16 within the fluid source 15 need to pass through the inlets 32 and oxidized particles 17 need to pass through the outlets 36.

A float 40 is connected to the frame 30 to provide buoyancy for the frame 30 so the frame 30 can stay afloat within the fluid source 15 of the reservoir structure 50a. In one embodiment, the float 40 provides just enough buoyancy so the carrier 20 is submerged within the fluid source 15 yet the upper wall 35 is positioned above the surface of the fluid source 15. The float 40 may comprise various types of foam or other floatable structures. The float 40 is tightly positioned within the lower receiver opening 33 and extends upwards to engage the lower surface of the upper wall 35.

In another embodiment, the float 40 comprises a heating source, which is primarily used to heat the fluid source 15 within the reservoir structure 50a during cold periods to prevent the fluid source 15 from freezing. Thus, the float 40 serves dual purposes; to keep the frame 30 afloat and heating the fluid source 15 to prevent freezing. In this embodiment, the upper receiver opening 37 is used, wherein the cord 41 from the heater configuration of the float 40 extends through the upper receiver opening 37 and the cord 31 includes a plug 42 which is electrically connected to an electrical socket to operate the heater comprised float 40.

As discussed previously, the reservoir structure 50a can be used to hold the fluid source 15 for livestock, wherein the fluid source 15 is water. However, the reservoir structure may take the form of various other embodiments, such as a plastic water bottle 50b, wherein the frame 30 may be omitted and the carrier 20 is simply wrapped around the inside perimeter of the bottle casing. Another embodiment shows the reservoir structure 50c comprises a toilet or urinal configuration and the carrier 20 simply positioned over the drain opening to disinfect fluid sources that come into contact with the carrier 20 within the urinal or toilet. Various other embodiments as discussed (e.g. cutting board, carrier 20 to clean up spills on a ground surface 12 such as an oil spill, etc.) may be used with the carrier 20. It is appreciated that the carrier 20 may be used for further embodiments, all of which require disinfection of a fluid source.

In use, the frame 30 including the carrier 20 is positioned within the fluid source 15 of the reservoir structure 50a so the lower wall 31 faces downward. The float 40 allows the carrier 20 and lower wall 31 to sink within the water either partially or wholly while keeping the upper wall 35 above the water surface so the oxidized particles 17 can more easily escape.

As the fluid source 15 including the organic contaminants 16 contacts the treatment surface 24, the oxygen from the fluid source 15 and the ultraviolet light from the light source 14 induce a chemical reaction with the photocatalyst 22 to form activated electrons which are necessary to form an antibacterial material (e.g. hydrogen peroxide). The antibacterial material generated from the photocatalytic reaction thus oxidizes the fluid source 15 including the contaminants 16 to disinfect the fluid source 15. The carrier 20 continues to operate as long as the carrier 20 is positioned at least partially within the fluid source 15 containing oxygen. As the chemical reaction takes place, the substrate material 21 slowly degrades. However, since the photocatalyst 22 is distributed evenly throughout the substrate material 21 the carrier 20 continually exposes a treatment surface 24 including the photocatalyst 22 and the substrate material 21 to the fluid source 15 and the light source 14.

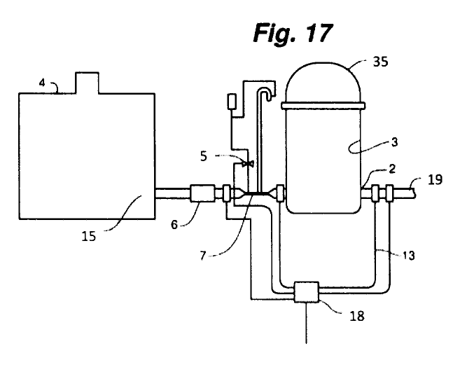

Example Embodiment of Improved Photocatalyst for Oxidation Reduction Chemistry as applied to a water purification system (See FIGS. 17-19):

Step 1) Reservoir Tank 4 begins filling with fluid source 15 including organic contaminants 16.

Step 2) The fluid proceeds out at a point near the Reservoir Tank's 4 bottom and flows past a check valve 6.

Step 3) Past the check valve 6 the fluid source 15 encounters an injector 7 where acid from an acidic reservoir 80 enters the stream along with air from a vent 81. The fluid then encounters a first pH probe which, with the help of a controller 18, meters the flow of acid via a pinch valve 5, which is under the control of the controller 18.

Step 4) The fluid source 15 then enters a treatment tank 3 and begins to support the float 40, positioning the frame 30 including the carrier 20 approximately 1 inch below the fluid surface. Light 10 entering at the top of the treatment tank 3 through the upper wall 35 irradiates the upper surface of the carrier 20 containing a treatment surface 24 including a photocatalyst 22 and the substrate material 21 where hydrogen ion and free oxygen unite to produce hydrogen peroxide. The hydrogen peroxide then begins to kill microorganisms; any unused hydrogen peroxide is returned to its constituent parts, water and free oxygen.

Step 5) Flow then continues on demand from the treatment tank 3 through the outlet opening 2 and outlet 19 user, through a filter to the user.

Note: When citric acid is used, excess citric acid in trace amounts is delivered to the user giving the final product a slight sour taste. Similar to rainwater, which if used as the stock water obviates the need for acidification. Some filtration will be necessary with the use of citric acid.

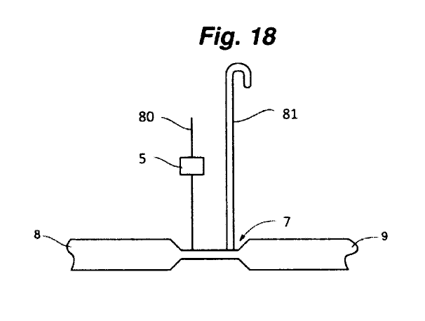

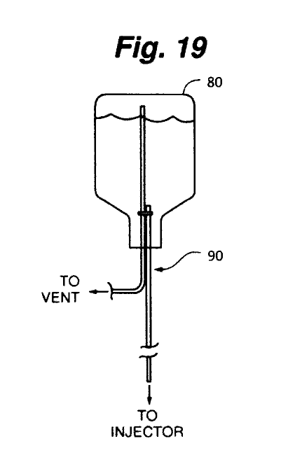

To further support this example embodiment, in FIG. 17, 13 represents electrical signal lines. In FIG. 18, 8 represents pipes from reservoir tank and 9 represents pipes to treatment tank. In FIG. 19, 90 represents a tube.

Example Embodiment of Improved Photocatalyst for Oxidation Reduction Chemistry as applied to a water purification system:

Step 1) User fills clear container containing an embodiment of the present disclosure with questionable water.

Step 2) User exposes the container to sunlight.

Step 3) User allows container to receive sunlight until the water gets cloudy.

Step 4) User filters now disinfected water. The water is now ready to drink.

### Method of Preparing a TiO₂ Photocatalyst

Two methods of preparing a titanium dioxide photocatalyst are disclosed. In one method, a saturated solution of catecholate ligand is prepared in a NaOH solution between a pH 8 and 12, at a temperature of between 60 to 100 degrees Celsius. Once the solution is at the appropriate temperature and acidic level, titanium isopropanol or titanium isobutanol may be added while agitating the solution. Then, the solution is allowed to precipitate, followed by decanting supemate. Acid solution is added to the remaining wet precipitate until achieving a pH of 3. The remaining wet precipitate is then filtered under suction. The filtered precipitate (filtrate) is washed with a pH 3 HCl solution. Finally, the filtrate is dried in an oven at ˜100 degrees Celsius for about between 3 to 12 hours.

In an alternative method, a saturated solution of catecholated ligand with a pH between 8 and 12 is brought to a temperature of ˜100 degrees Celsius. Titanium dioxide anatase nanoparticles are slowly added to the solution with agitation. The solution is then allowed to precipitate, followed by decanting supernate. Acid solution is added to the remaining wet precipitate until achieving a pH of 3. Then the remaining wet precipitate is filtered under suction. The filtered precipitate (filtrate) is washed with pH 3 HCl acid solution. The filtrate is then dried in an oven at ˜100 degrees Celsius for about between 3 to 12 hours.

Method of Producing a Dye Sensitized TiO₂ Photocatalyst Surface over a Porcelain Substrate

Methods of producing a dye sensitized titanium dioxide photocatalyst surface over a porcelain substrate are disclosed. In one method, anatase titanium dioxide photocatalyst impregnated low temperature glaze is applied to the porcelain substrate. In the alternative to applying a titanium dioxide photocatalyst impregnated low temperature glaze, a high titanium dioxide coating solution may be applied instead. The porcelain substrate is then fired in a kiln to cure the clay and glaze. Optionally, after firing, etching solution may be applied to the cured titanium dioxide photocatalyst impregnated low temperature glaze. The glaze is then washed in an acid bath. The glazed porcelain substrate is then heated to about 100 degrees Celsius in a saturated pH 10 Sodium Hydroxide Solution of Azo Dye for about 24 hours. The pH of the solution may range between pH 8-12 and, in this example, is a pH of 10. Finally, the glazed porcelain substrate is rinsed with distilled water.

### Dye Sensitized TiO₂ Photocatalyst

The titanium dioxide photocatalyst independently is capable of absorbing light only in the Ultraviolet frequencies around 200 nm. The inability of TiO₂ photocatalyst to absorb other spectrums of light negatively impacts the total energy absorbed by the photocatalyst and the net result of the photocatalytic reaction. This creates a large inefficiency in titanium dioxide photocatalyst based reactions. However, through experimentation it has been ascertained that by bonding titanium dioxide photocatalyst with Azo Dye, the dye associates itself with the photocatalyst's ability to absorb light. The result is a dye sensitized TiO₂ photocatalyst capable of absorbing a broader spectrum of light, including: Ultraviolet (˜200nm) and Broad Visible (up to 500 nm). The two combined resulting energy level excitations result in a 30% increase in light absorption over TiO₂ photocatalyst alone.

In one embodiment, the carrier 20 is comprised of a substrate material 21 and a dye sensitized photocatalyst 22 material distributed evenly throughout. The treatment surface 24 includes the portion of the carrier which has the dye sensitized photocatalyst 22 distributed throughout the substrate material 21. The treatment surface 24 and dye sensitized photocatalyst 22 can be distributed evenly throughout the entire substrate material 21 and thus entire carrier 20 as illustrated in FIG. 7. However, in alternate embodiments, the treatment surface 23 may be instead along the perimeter walls of openings extending through the carrier 20 (in the mesh shape), upon a top surface, a bottom surface, or portions thereof. The treatment surface 24 may simply be a small portion of the substrate material 21 or carrier 20, of which contacts the fluid source 15 and receives the ultraviolet and broad visible light from the light source 14. The substrate material 21 may also comprise a permeable and absorbent structure so the contaminants 16 can travel within the carrier 20 to be oxidized within. It is appreciated various combinations of the above described, as well as other combinations, may also be used to combine the dye sensitized photocatalyst 22 with the substrate material 21.

The substrate material 21 is pigmented with the dye sensitized photocatalyst 22 which can be comprised of Azo dye and titanium dioxide, and has properties to induce a chemical reaction when exposed to ultraviolet and broad visible light rays from the light source 14. The dye sensitized photocatalyst 22 further can comprise dye sensitized titanium dioxide in the anatase crystalline form rather than its rutile form. After the pigmentation melt process, the substrate material 21 impregnated with dye sensitized photocatalyst 22 can be extruded in various forms whose surfaces 24 are photocatalytic in the oxidation of oxygenated water (e.g. fluid source 15) to hydrogen peroxide.

The dye sensitized photocatalyst 22 comprises an absorbing substance to be able to absorb the ultraviolet and broad visible light. When receiving the ultraviolet and broad visible light the dye sensitized photocatalyst 22 is able to oxidize the organic contaminants 16 to essentially self-disinfect the fluid source 15 or other type of surface or object. The treatment surface 24 extends throughout the carrier 20 and thus is continually exposed as substrate material 21 degrades away from the chemical reaction of the oxygen from the fluid source 15 and the ultraviolet and broad visible light from the light source 14 to form activated electrons allowing for the creation of hydrogen peroxide to break down the contaminants 16 into oxidized particles 17 as illustrated in FIG. 8. As the fluid source 15 including the organic contaminants 16 contacts the treatment surface 24, the oxygen from the fluid source 15 and the ultraviolet and broad visible light from the light source 14 induce a chemical reaction with the dye sensitized photocatalyst 22 to form an antibacterial material (e.g. hydrogen peroxide). The antibacterial material generated from the photocatalytic reaction thus oxidizes the fluid source 15 including the contaminants 16 to disinfect the fluid source 15.

The carrier 20 continues to operate as long as the carrier 20 is positioned at least partially within the fluid source 15 containing oxygen. As the chemical reaction takes place, the substrate material 21 slowly degrades. However, since the dye sensitized photocatalyst 22 is positioned evenly throughout the substrate material 21 the carrier 20 continually exposes a treatment surface 24 including the photocatalyst 22 and the substrate material 21 to the fluid source 15 and the light source 14.

The dye sensitized TiO₂ photocatalyst embodiment of this disclosure is also amenable to the various embodiments mentioned previously and all their various modifications that are obvious to one skilled in the art.

The preceding description has been presented only to illustrate and describe various examples or illustrations of the embodiments. It is not intended to be exhaustive or limit to any precise form disclosed. Many modifications and variations are possible in light of the above teaching. Although specific examples have been illustrated and described herein, it will be appreciated by those of ordinary skill in the art that any arrangement calculated to achieve the same purpose could be substituted for the specific examples shown. This application is intended to cover adaptations or variations of the present subject matter. Thus, particular embodiments of the subject matter have been described. Other embodiments are within the scope of the following claims.

What is claimed is:

1. A disinfectant system, comprising:
a light source for producing ultraviolet light;
a fluid source including organic contaminants within; and
a carrier comprising a substrate material, a photocatalyst, and a treatment surface;
said substrate material comprising an electrically non conductive and degradable material, said photocatalyst evenly distributed throughout said substrate material and said treatment surface, said treatment surface disposed within said fluid source and adapted to continually expose said substrate material and said photocatalyst to said fluid source and said light source, said treatment surface adapted to continuously regenerate as said substrate material degrades.

2. The disinfectant system of claim 1, wherein said substrate material is selected from the group consisting of plastic, rubber, and glass, and combinations thereof.

3. The disinfectant system of claim 1, wherein said photocatalyst comprises titanium dioxide.

4. The disinfectant system of claim 3, wherein said titanium dioxide is in an anatase form.

5. The disinfectant system of claim 1, wherein said substrate material comprises a plastic and wherein said photocatalyst comprises titanium dioxide in an anatase form.

6. The disinfectant system of claim 1, including:
a frame positioned within said fluid source; and
a float connected to said frame, wherein said float maintains said frame at least partially buoyant within said fluid source;
wherein said carrier is connected to said frame, so said carrier is suspended at least partially below a fluid surface of said fluid source.

7. The disinfectant system of claim 6, wherein said frame includes a lower wall including a plurality of inlets for receiving said organic contaminants and an upper wall including a plurality of outlets for releasing said oxidized organic contaminants, wherein said upper wall is spaced apart from said lower wall.

8. The disinfectant system of claim 7, wherein said carrier is connected to said lower wall between said lower wall and said upper wall.

9. The disinfectant system of claim 1, wherein said carrier comprises a mesh shaped structure.

10. A disinfectant system, comprising:
a frame having a lower wall including at least one inlet and an upper wall including at least one outlet, wherein said lower wall is vertically offset with respect to said upper wall;
wherein at least said upper wall of said frame is transparent;
a float connected to said frame for providing buoyancy to said frame; and
a carrier connected to said lower wall between said lower wall and said upper wall, wherein said upper wall substantially surrounds an upper surface of said carrier;
wherein said carrier induces a photocatalytic reaction;
wherein said carrier is in fluid communication with said at least one inlet for receiving organic contaminants from a fluid source;
wherein said carrier is in fluid communication with said at least one outlet for releasing a plurality of oxidized particles generated during said photocatalytic reaction.

11. The disinfectant system of claim 10, wherein said carrier comprises a substrate material and a photocatalyst, wherein said photocatalyst is evenly distributed throughout said substrate material.

12. The disinfectant system of claim 11, wherein said substrate material comprises a plastic and wherein said photocatalyst comprises titanium dioxide in an anatase form.

13. The disinfectant system of claim 10, wherein said float comprises a heating source.

14. The disinfectant system of claim 10, wherein said carrier comprises a mesh shaped structure.

15. A method for disinfecting a fluid source comprising the steps of:
introducing a disinfectant system in a fluid source including organic contaminants, the disinfectant system comprising a carrier comprising a substrate material, a photocatalyst and a treatment surface, wherein said photocatalyst is configured to be evenly distributed throughout said substrate material, such that as the photocatalyst degrades, the treatment surface regenerates to expose new photocatalytic material, allowing for a continued reaction;
exposing the disinfectant system continually to a light source; and
oxidizing said fluid source's organic contaminants, disinfecting the fluid source.

16. A disinfectant system, comprising:
a light source for producing ultraviolet and visible light;
a fluid source including organic contaminants within; and
a carrier comprising a substrate material, a dye sensitized photocatalyst, and a treatment surface;
said substrate material comprising an electrically non conductive and degradable material, said dye sensitized photocatalyst evenly distributed throughout said substrate material and said treatment surface, said treatment surface disposed within said fluid source and adapted to continually expose said substrate material and said dye sensitized photocatalyst to said fluid source and said light source, said treatment surface adapted to continuously regenerate as said substrate material degrades.

17. A method of preparing a titanium dioxide photocatalyst comprising:
preparing a saturated solution of a catecholate ligand of choice in a NaOH solution at a pH between 8 and 12 and temperature of between about 60-100° C.;
adding dropwise: titanium isopropanol or titanium isobutanol, with agitation;
settling the saturated solution;
decanting a supernate;
adjusting a remaining wet precipitate to pH 3 with acid solution;
filtering the precipitate under suction;
washing a filtrate with pH 3 HCl solution; and
drying the filtrate in an oven at about 100° C. between approximately 3-12 hours.

18. A method of preparing a titanium dioxide photocatalyst comprising:
preparing a saturated solution of catecholate ligand at pH 8-12 and about 100° C.;
adding titanium dioxide anatase nanoparticles slowly with agitation to the saturated solution;
settling the saturated solution;
decanting a supernate;
adjusting a remaining wet precipitate to pH 3 with acid solution;
filtering the precipitate under suction;
washing a filtrate with pH 3 HCl solution; and
drying filtrate in an oven at about 100° C. between about 3-12 hours.

19. A method of applying a dye sensitized titanium dioxide photocatalyst layer to a porcelain substrate comprising:
applying an anatase titanium dioxide photocatalyst impregnated low temperature glaze to the porcelain substrate;
firing the porcelain substrate, thereby curing the anatase titanium dioxide photocatalyst impregnated low temperature glaze to the porcelain substrate;
washing the cured titanium dioxide photocatalyst impregnated low temperature glaze in an acid bath;
heating the cured titanium dioxide photocatalyst impregnated low temperature glaze to about 100° C. in a saturated pH 8-12 solution Sodium Hydroxide Solution of Azo Dye for about 24 hours; and
rinsing the glazed porcelain substrate in distilled water.

20. The method of claim 19, wherein between the firing and soaking steps, etching solution is applied to the cured titanium dioxide photocatalyst impregnated low temperature glaze.

21. The method of claim 19, wherein alternative to applying an anatase titanium dioxide photocatalyst impregnated low temperature glaze, a high titanium dioxide coating solution is applied.
